SEQUENCE LISTING (28 sequences)

SEQ ID NO: 1 - protein, 13 aa, synthetic construct
VCAVGLWGNWLVM

SEQ ID NO: 2 - protein, 14 aa, synthetic construct
LEKFVKYESIVGKV

SEQ ID NO: 3 - protein, 9 aa, synthetic construct
LFIVYAYMY

SEQ ID NO: 4 - protein, 6 aa, synthetic construct
YPWFYL

SEQ ID NO: 5 - protein, 6 aa, synthetic construct
LYAAYL

SEQ ID NO: 6 - protein, 5 aa, synthetic construct
LYAYL

SEQ ID NO: 7 - protein, 10 aa, synthetic construct
DTCIVCLCCL

SEQ ID NO: 8 - protein, 11 aa, synthetic construct
KVCIDYLICLA

SEQ ID NO: 9 - protein, 12 aa, synthetic construct
NPVLYVCVLQLL

SEQ ID NO: 10 - protein, 13 aa, synthetic construct
AAIKYCICFIIVC

SEQ ID NO: 11 - protein, 10 aa, synthetic construct
VAILVLLLRD

SEQ ID NO: 12 - protein, 9 aa, synthetic construct
LVVLLILLD

SEQ ID NO: 13 - protein, 13 aa, synthetic construct
  SITE 1: note = D-methionine
  SITE 2: note = D-Valine
  SITE 3: note = D-Leucine
  SITE 4: note = D-Tryptophan
  SITE 5: note = D-Asparagine
  SITE 6: note = D-Glycine
  SITE 7: note = D-Tryptophan
  SITE 8: note = D-Leucine
  SITE 9: note = D-Glycine
  SITE 10: note = D-Valine
  SITE 11: note = D-Alanine
  SITE 12: note = D-Cysteine
  SITE 13: note = D-Valine
MVLWNGWLGVACV

SEQ ID NO: 14 - protein, 6 aa, synthetic construct
  SITE 1: note = D-Leucine
  SITE 2: note = D-Tyrosine
  SITE 3: note = D-Phenylalanine
  SITE 4: note = D-Tryptophan
  SITE 5: note = D-Proline
  SITE 6: note = D-Tyrosine
LYFWPY

SEQ ID NO: 15 - protein, 6 aa, synthetic construct
  SITE 1: note = D-Leucine
  SITE 2: note = D-Tyrosine
  SITE 3: note = D-Phenylalanine
  SITE 4: note = D-Tryptophan
  SITE 5: note = D-Proline
  SITE 6: note = D-Tyrosine
LYFWPY

SEQ ID NO: 16 - protein, 11 aa, synthetic construct
YPWFLTREKSQ

SEQ ID NO: 17 - protein, 11 aa, synthetic construct
  SITE 1: note = D-Glutamine
  SITE 2: note = D-Serine
  SITE 3: note = D-Lysine
  SITE 4: note = D-Glutamic acid
  SITE 5: note = D-Arginine
  SITE 6: note = D-Threonine
  SITE 7: note = D-Leucine
  SITE 8: note = D-Phenylalanine
  SITE 9: note = D-Tryptophan
  SITE 10: note = D-Proline
  SITE 11: note = D-Tyrosine
QSKERTLFWPY

SEQ ID NO: 18 - protein, 16 aa, synthetic construct
LAIAITALYSYPWFYL

SEQ ID NO: 19 - protein, 16 aa, synthetic construct
  SITE 11: note = D-Leucine
  SITE 12: note = D-Tyrosine
  SITE 13: note = D-Phenylalanine
  SITE 14: note = D-Tryptophan
  SITE 15: note = D-Proline
  SITE 16: note = D-Tyrosine
LAIAITALYSLYFWPY

SEQ ID NO: 20 - protein, 5 aa, synthetic construct
  SITE 1: note = D-Leucine
  SITE 2: note = D-Tyrosine
  SITE 3: note = D-Alanine
  SITE 4: note = D-Tyrosine
  SITE 5: note = D-Leucine
LYAYL

SEQ ID NO: 21 - protein, 5 aa, synthetic construct
  SITE 3: note = N-(3beta-Hydroxy-5-cholenoyl)ornithine
LYXYL

SEQ ID NO: 22 - protein, 6 aa, synthetic construct
  SITE 6: note = N-(3beta-Hydroxy-5-cholenoyl)ornithine
YPWFLX

SEQ ID NO: 23 - protein, 7 aa, synthetic construct
  SITE 1: note = N-(3beta-Hydroxy-5-cholenoyl)ornithine
XIVYAYL

SEQ ID NO: 24 - protein, 7 aa, synthetic construct
  SITE 4: note = N-(3beta-Hydroxy-5-cholenoyl)ornithine
LYAXAYL

SEQ ID NO: 25 - protein, 13 aa, synthetic construct
  SITE 13: note = N-(3beta-Hydroxy-5-cholenoyl)ornithine
ITALYSKVKKVGX

SEQ ID NO: 26 - protein, 17 aa, synthetic construct
  SITE 17: note = N-(3beta-Hydroxy-5-cholenoyl)ornithine
LAIAITALYSKVSKVGX

SEQ ID NO: 27 - protein, 17 aa, synthetic construct
  SITE 17: note = N-(3beta-Hydroxy-5-cholenoyl)ornithine
LAIAITALYSIVLIVGX

SEQ ID NO: 28 - protein, 14 aa, synthetic construct
  SITE 14: note = N-(3beta-Hydroxy-5-cholenoyl)ornithine
KIMKCSICLGLFGX

(19) United States
(12) Patent Application Publication
(10) Pub. No.: US 2025/0215051 A1
(43) Pub. Date: Jul. 3, 2025

(54) Transmembrane Allosteric Short Peptides Modulating Human Mu-Opioid Receptor for Analgesic Effects and Covalent Steroid Modifications of Transmembrane Allosteric Peptides for Enhanced Mu-Opioid Receptor Interaction in Chronic Pain Therapies

(71) Applicant: Mu42 SPOLKA Z OGRANICZONA ODPOWIEDZIALNOSCIA, Rzeszow (PL)

(72) Inventors: Kamil Mieszkowski, Warszawa (PL); Maciej Ciemny, Warszawa (PL)

(73) Assignee: Mu42 SPOLKA Z OGRANICZONA ODPOWIEDZIALNOSCIA, Rzeszow (PL)

(21) Appl. No.: 19/003,021

(22) Filed: Dec. 27, 2024

Related U.S. Application Data
(60) Provisional application No. 63/615,955, filed on Dec. 29, 2023.

(51) Int. Cl. C07K 7/06; C07K 7/08; G16B 15/30

(52) U.S. Cl. CPC C07K 7/06; C07K 7/08; G16B 15/30

(57) ABSTRACT

The aspects of the disclosed embodiments introduce a novel class of bioactive compounds, including short peptides and peptidosteroids, designed for the selective modulation of G-Protein Coupled Receptors (GPCRs), with a particular focus on the human mu-opioid receptor (MOR). These compounds feature specific amino acid sequences and modifications such as acylation and covalent steroid modifications, including 3beta-hydroxy-5-cholenoic acid or other fatty acid derivatives. These modifications enhance stability, membrane affinity, receptor selectivity, and allosteric modulation potential. The aspects of the disclosed embodiments provide a platform for receptor-targeted therapies, particularly for chronic pain conditions such as neuropathic pain, cancer-related pain, and postoperative pain. By biasing MOR signaling toward analgesic pathways and reducing adverse effects, these compounds address the limitations of traditional opioid therapies, offering improved safety, efficacy, and bioavailability. The described peptides and peptidosteroids also exhibit potential for broader GPCR-mediated therapeutic applications, establishing their utility in advancing precision pharmacology.

### CROSS-REFERENCE TO RELATED APPLICATIONS

This application claims priority to, and the benefit of, U.S. Provisional Patent Application Ser. No. 63/615,955 filed on 29 Dec. 2023, the disclosure of which is incorporated herein by reference in its entirety.

### INCORPORATION BY REFERENCE

The sequence listing in XML format is incorporated herein by reference in its entirety.
a. Name of File: 22311_2024-12-27_sequence
b. Date of Creation: 27 Dec. 2024
c. Size of File: 44,587 Bytes

### BACKGROUND

Pain management is a critical area of healthcare that has long relied on opioids targeting the mu-opioid receptor (MOR). While effective for pain relief, these opioids are associated with significant drawbacks, including adverse effects like drowsiness, constipation, respiratory depression, and the potential for addiction. The societal consequences of opioid misuse, including addiction, overdoses, and healthcare strain, have fueled the ongoing opioid crisis, highlighting the urgent need for safer alternatives.

G Protein-Coupled Receptors (GPCRs), a family of approximately 800 proteins in the human genome, are central to human physiology, converting extracellular signals into physiological responses (1). These receptors mediate various processes through interaction with hormones, neurotransmitters, and chemokines, making them pivotal targets for therapeutic intervention. Drugs targeting GPCRs represent roughly one-third of the global therapeutic drug market, underscoring their importance (2). However, the development of GPCR-targeted drugs has primarily focused on orthosteric ligands, which interact with highly conserved active sites. This approach often results in poor selectivity and unintended off-target effects.

Recent advances in structural biology and receptor pharmacology have shifted attention to allosteric modulation. Allosteric sites, less conserved across GPCR subfamilies, offer an opportunity for designing highly selective drugs. Modulation through these sites can achieve biased signaling, enhancing therapeutic efficacy while reducing adverse effects (3,4). Additionally, allosteric modulators maintain natural signaling rhythms (5,6) and exhibit a “ceiling effect,” providing improved safety in overdose scenarios (5,7,8).

The aspects of the disclosed embodiments focus on the development of short peptides designed to allosterically modulate the activity of the human mu-opioid receptor (MOR) by binding to its transmembrane region. This approach aims to elicit analgesic effects while addressing the limitations and challenges associated with conventional opioid-based therapies.

Our work addresses also the need of further optimizing the peptide sequence by focusing on a novel class of peptidomimetics designed to target allosteric sites on GPCRs, particularly the mu-opioid receptor. These peptidomimetics leverage the unique characteristics of intramembrane binding sites, utilizing peptides' larger contact surfaces to achieve high selectivity and reduced side effects.

The transmembrane allosteric peptides offer several advantages. Their relatively large contact surface area with the target receptor enhances selectivity, potentially reducing side effects and toxicity commonly observed with traditional opioids. Leveraging the endogenous role of peptides as mediators of protein-protein interactions, our approach aligns with the design of highly selective receptor ligands and allosteric modulators.

While the design of these peptidomimetics was informed by advanced in silico modeling techniques emphasizing receptor dynamics, the aspects of the disclosed embodiments lie in the molecules themselves. By incorporating dynamic descriptors into the design process, we have achieved a breakthrough in peptidomimetic efficacy and selectivity, moving beyond traditional structure-based approaches that neglect receptor dynamics.

In summary, the aspects of the disclosed embodiments offer a novel therapeutic strategy for pain management by combining the advantages of allosteric modulation with the precision of peptide-based drug design. By addressing the limitations of existing opioid therapies, these peptidomimetics provide a pathway to safer and more effective treatments, ultimately benefiting patients and alleviating the societal burden of opioid-related challenges.

### BRIEF SUMMARY

The aspects of the disclosed embodiments introduce a distinct class of short transmembrane peptides and their derivatives featuring covalent steroid modifications known as peptidosteroids, specifically designed for enhanced interaction with the mu-opioid receptor (MOR) in the process known as in silico target-based ab initio candidate design. This distinct class of analgetic peptides and their derivatives includes N-acylation of ornithine or lysine residues and N-terminal acylation with 3beta-hydroxy-5-cholenoic acid, decanoic acid, lauric acid, myristoyl, palmitoyl, and stearic acid and respective modification of the C-terminus. These modifications confer unique physicochemical properties, enhancing membrane and receptor binding, allosteric modulation of the target receptor and improving therapeutic potential.

These peptides and their modifications-peptidosteroids, characterized by a unique set of sequences and chemical modifications, demonstrate distinct structural properties facilitating effective interactions with the plasma membrane and the mu-opioid receptor (MOR).

Transmembrane peptides and peptidosteroids represent a significant advancement in the field of receptor-targeted therapeutics, offering a potent and selective approach to allosterically modulate the mu-opioid receptor. Their design is rooted in a deep understanding of peptide-membrane and peptide-receptor interactions, leveraging covalent steroid modifications to achieve desired pharmacological profiles.

The aspects of the disclosed embodiments pertain to an innovative class of short peptides and their derivatives designed for the allosteric modulation of the human mu-opioid receptor (MOR). This expanded range of compounds includes not only peptides composed of all-natural amino acids but also those incorporating D-amino acids, retro-inverted sequences, and various N- and C-terminal modifications.

The central innovation in the peptide derivatives lies in the incorporation of 3beta-hydroxy-5-cholenoic acid, decanoic acid, lauric acid, myristoyl, palmitoyl, and stearic acid moieties. These modifications significantly enhance the hydrophobicity and amphiphilic nature of the peptidosteroids, leading to improved stability in membrane and a stronger affinity for the mu-opioid receptor transmembrane binding region. Such enhancements are pivotal for augmenting the therapeutic efficacy of these compounds, particularly in the context of chronic pain management.

The molecules presented were designed computationally using proprietary computational program designed by the authors. The input for the procedure is structural data on the allosteric site of planned interaction. This aspects of the disclosed embodiments pave the way for a new class of analgesics that exhibit increased selectivity, reduced side effects, and an improved safety profile, addressing the limitations of traditional opioid-based therapies. As such, peptidosteroids stand at the forefront of innovative strategies in pain management, holding promise for revolutionizing the treatment of chronic pain conditions.

### BRIEF DESCRIPTION OF DRAWINGS

FIG. 1: The figure depicts a schematic representation of a peptide with specific terminal modifications. In the centre is a ribbon illustration of a peptide sequence, indicating a chain of amino acids, the possible sequences are listed in Table 1. This peptide chain is flanked by two boxes, each representing different types of modifications at the peptide's ends, including n- and c-terminal modifications.

FIG. 2 provides a schematic representation of a modified peptide molecule, illustrating its structural components and the location of specific modifications. At the center of the diagram is a depiction of the peptide, rendered in a ribbon-like format to emphasize the peptide backbone and its spatial conformation. This visualization highlights the sequence of amino acids, with each residue clearly labeled for clarity and scientific precision. The n-th residue, which may be either ornithine or lysine, is explicitly marked as R_n, indicating its position within the sequence. Additionally, the modification site on this residue is annotated with an attached steroid acid moiety, denoted as X, positioned above the corresponding amino acid side chain.

FIG. 3 presents a dose-activity chart for the invented substance, derived from in vitro experiments conducted in the presence of the reference MOR agonist DAMGO at an EC20 concentration. The chart values are normalized, with 0% corresponding to the activity of the reference agonist at EC20 and 100% representing the maximum activity of the reference agonist. The data highlights a critical and unexpected feature of the aspects of the disclosed embodiments, demonstrating a logEC50 (M) value in the sub-5 range. This result underscores the high potency of the aspects of the disclosed embodiments, as it achieves significant activity at low concentrations.

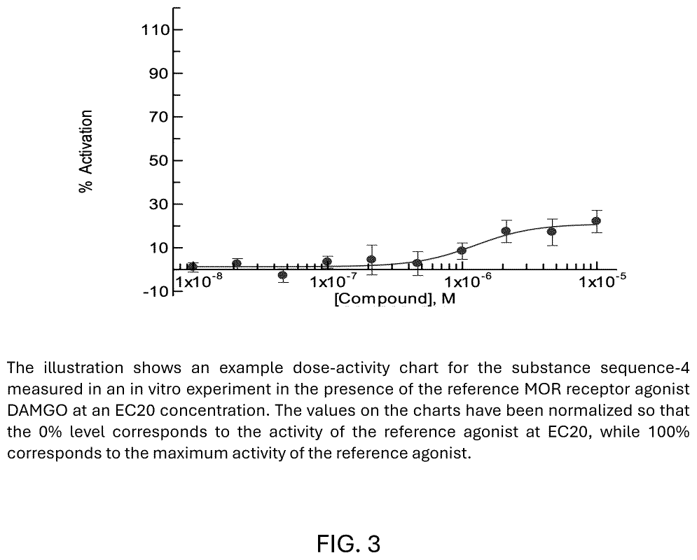

### DETAILED DESCRIPTION

This aspects of the disclosed embodiments relate to a novel class of bioactive molecules, termed transmembrane peptides and their derivatives-peptidosteroids, specifically designed to modulate the mu-opioid receptor (MOR) with high selectivity. These molecules constitute a significant advancement in the field of opioid-based therapies by offering improved safety and efficacy, achieved through innovative structural modifications and precise receptor-targeting mechanisms.

The aspects of the disclosed embodiments pertain to an innovative class of short peptides and peptidomimetics designed for the allosteric modulation of the human mu-opioid receptor (MOR). This expanded range of compounds includes not only peptides composed of all-natural amino acids but also those incorporating D-amino acids, retro-inverted sequences, and various N- and C-terminal modifications. Peptidosteroids are characterized by covalent steroid modifications that enhance their therapeutic potential. These modifications include N-terminal acylation with 3beta-hydroxy-5-cholenoic acid or alternative fatty acids, such as decanoic acid, lauric acid, myristoyl, palmitoyl, or stearic acid. The acylation sites are strategically located on the side chains of ornithine or lysine residues, or at the peptide's N-terminus. These modifications confer increased hydrophobicity, amphiphilicity, and structural integrity to the molecules, enabling efficient interaction with the plasma membrane and the hydrophobic intramembrane regions of MOR. This targeted interaction ensures optimal receptor engagement and functional modulation, which are critical for achieving the desired pharmacological effects.

For clarity and consistency with emerging scientific consensus, peptides and their derivatives are herein defined as polymeric linear molecules comprising fewer than fifty amino acids, either natural or modified (9). This definition serves to delineate peptides from larger biologics such as proteins, which occupy a distinct domain in pharmaceutical applications.

This aspects of the disclosed embodiments introduce a new approach to receptor-targeted drug design by leveraging intramembrane binding sites of MOR, a biologically validated but underexploited mechanism of receptor modulation. Intramembrane interactions play a pivotal role in stabilizing specific conformational states of G-protein coupled receptors (GPCRs) and regulating downstream signaling pathways. Despite their established biological importance, these hydrophobic binding regions have remained largely unutilized in drug development, primarily due to significant technological challenges. By addressing these challenges, the aspects of the disclosed embodiments establish peptidosteroids as a novel therapeutic platform, offering a pathway to safer and more effective opioid-based treatments.

The challenges addressed by the aspects of the disclosed embodiments include the inherent complexity of accurately modeling receptor-ligand interactions within the highly dynamic lipid bilayer environment. The fluid and heterogeneous nature of the membrane creates significant obstacles for traditional computational and experimental methods, particularly in capturing the transient and entropy-driven interactions that characterize intramembrane binding sites. Conventional approaches often fail to account for the intricate conformational changes and localized dynamics that are critical for understanding and optimizing such interactions.

This aspects of the disclosed embodiments overcome these challenges through the rational design of peptidosteroids. In contrast to traditional opioid therapies, which primarily target the highly conserved orthosteric sites of the mu-opioid receptor (MOR), peptidosteroids are engineered to interact with less-conserved regions of the receptor structure. By focusing on these intramembrane binding sites, the peptidosteroids achieve greater selectivity and reduced off-target effects, representing a significant departure from existing pharmacological strategies.

Table 1 below lists the sequences of these peptides, each designed to elicit analgesic effects with reduced side effects. The sequences are presented in one letter amino acid code, uppercase letters are used for L-amino acids while lowercase letters are used for D-amino acids. For each of the sequences, additionally all of the following modifications are included: n-terminal acylation, c-terminal amidation, n-terminal lipid derivatives of varying polycarbon chain lengths up to palmitoyl modification. Experimental data (not shown) demonstrates the efficacy of these peptides in positively modulating MOR activity while exhibiting enhanced safety profiles in overdose situations. These peptides hold promise for chronic pain therapy, offering potential relief for conditions such as neuropathic pain, cancer-related pain, and postoperative pain, while minimizing the societal challenges associated with opioid abuse.

sequence-1
VCAVGLWGNWLVM
sequence-2
LEKFVKYESIVGKV
sequence-3
LFIVYAYMY
sequence-4
YPWFYL
sequence-5
LYAAYL
sequence-6
LYAYL
sequence-7
DTCIVCLCCL
sequence-8
KVCIDYLICLA
sequence-9
NPVLYVCVLQLL
sequence-10
AAIKYCICFIIVC
sequence-11
VAILVLLLRD
sequence-12
LVVLLILLD
sequence-13
mvlwngwlgvacv
sequence-14
lyfwpy
sequence-15
lyfwpy
sequence-16
YPWFLTREKSQ
sequence-17
qskertlfwpy
sequence-18
LAIAITALYSYPWFYL
sequence-19
LAIAITALYSlyfwpy
sequence-20
lyayl

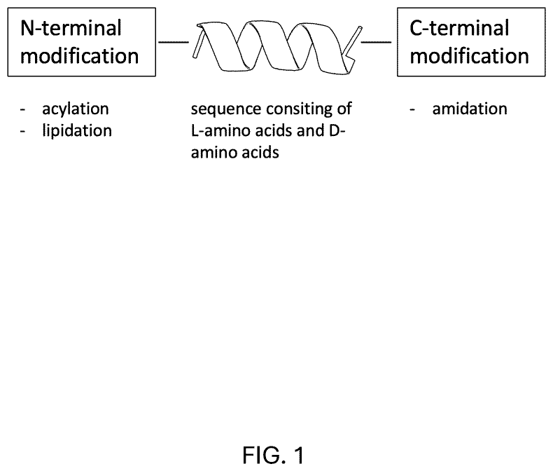

FIG. 1: The figure depicts a schematic representation of a peptide with specific terminal modifications. In the centre is a ribbon illustration of a peptide sequence, indicating a chain of amino acids, the possible sequences are listed in Table 1. This peptide chain is flanked by two boxes, each representing different types of modifications at the peptide's ends, including n- and c-terminal modifications.

The table 2 presents example squences of the peptidosteroids presenting possible ways of covalently modifying the peptide sequence to achieve a peptidosteroid molecule (sequence numbering is continuous with table 1 for clarity):

sequence-21
LY{ORN(3BETA-HYDROXY-5-CHOLENOICACID)}YL
sequence-22
YPWFL{ORN(3BETA-HYDROXY-5-CHOLENOICACID)}
sequence-23
{ORN(3BETA-HYDROXY-5-CHOLENOICACID)}IVYAYL
sequence-24
LYA{ORN(3BETA-HYDROXY-5-CHOLENOICACID)}AYL
sequence-25
ITALYSKVKKVG{ORN(3BETA-HYDROXY-5-
CHOLENOICACID)}
sequence-26
LAIAITALYSKVSKVG{ORN(3BETA-HYDROXY-
5-CHOLENOICACID)}
sequence-27
LAIAITALYSIVLIVG{ORN(3BETA-HYDROXY-5-
CHOLENOICACID)}
sequence-28
KIMKCSICLGLFG{ORN(3BETA-HYDROXY-5-
CHOLENOICACID)}

In these sequences both ORN (ornithine) may be replaced with lysine and the steroid group 3BETA-HYDROXY-5-CHOLENOIC ACID may be replaced with other steroid-group based acid molecule. In addition to these modifications, the sequences are also modified with N-terminal acylation with 3beta-hydroxy-5-cholenoic acid or alternative fatty acids: decanoic acid, lauric acid, myristoyl, palmitoyl, or stearic acid.

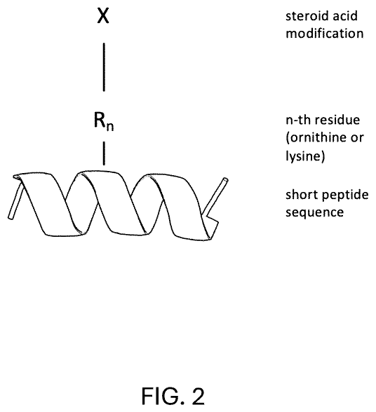

FIG. 2: The figure presents a schematic representation of a modified peptide molecule. At the centre is a depiction of a peptide with a short amino acid sequence, shown as a ribbon-like structure to represent the peptide backbone. The n-th residue (ornithine or lysine) is marked (as R_n) and its steroid acid moiety modification is presented as X above.

Specifically, for the peptides listed above, the maximum positive allosteric modulation activity was observed in the presence of DAMGO and in the presence of morphine, and the activity exceeds the 20% threshold for positive activity normalized to the EC20 level of the DAMGO agonist. The observed maximum activation effect was 40.30% when normalized to EC100 DAMGO.

The synthesis of peptidosteroids has been developed to preserve the structural integrity and biological activity of the peptide backbone while seamlessly incorporating the requisite functional groups. The process employs secondary amide group acylation under mild reaction conditions, ensuring that the modifications are both efficient and non-disruptive to the delicate peptide structure. Furthermore, this synthesis approach is scalable, allowing for the production of peptidosteroids in quantities sufficient for both research and clinical applications, thereby enabling their practical deployment as innovative therapeutic agents.

The design process of the aspects of the disclosed embodiments was guided by the application of advanced computational methodologies, including molecular dynamics simulations and structural modeling techniques. These approaches were instrumental in providing detailed insights into the dynamic behavior of the mu-opioid receptor (MOR), particularly its intramembrane regions. By simulating the receptor's interactions within a lipid bilayer environment, the analysis identified critical residues involved in binding and signaling. These residues, located both on the protein surface and within its structure, were found to align closely with established binding sites for transmembrane ligands. Notably, these include interactions with membrane components, such as cholesterol, a known allosteric modulator of GPCRs (10-16), and membrane-associated proteins like receptor activity modifying proteins (RAMPs) (17,18).

By integrating these computational findings, the peptidosteroids were designed to achieve selective binding at these identified sites, optimizing their therapeutic efficacy. Molecular dynamics simulations further revealed that the peptidosteroids exhibit a remarkable capacity for dynamic conformational adaptation. Specifically, the peptides transition from a folded state in aqueous environments—where hydrophobic residues are shielded by hydrophilic segments—to an extended conformation upon interaction with the receptor's hydrophobic membrane environment. This conformational flexibility is critical, as it enables the peptides to engage efficiently with the MOR, stabilizing receptor conformations that favor therapeutic signaling. Such dynamic adaptability underscores the innovation of the peptidosteroid design, ensuring precise and effective modulation of the receptor's activity.

A comprehensive library of representative peptidosteroid sequences was developed, each specifically designed to facilitate targeted interactions with the mu-opioid receptor (MOR). These sequences were systematically modified at both the amino acid backbone and side chains, incorporating steroidal groups to enhance receptor binding and modulate activity effectively. The design process ensured that each sequence was optimized for selective engagement with MOR, emphasizing functional specificity and improved therapeutic potential.

The properties and functionality of these peptidosteroids were validated using standard experimental methods. To evaluate their allosteric modulation capabilities, functional cellular assays were conducted on CHO-K1 cell lines expressing MOR. These assays utilized fluorescent techniques to measure intracellular cAMP levels as a marker of receptor activity.

The peptidosteroids were tested in co-administration with orthosteric ligands, such as morphine and DAMGO, to assess their ability to influence receptor signaling.

The results demonstrated detectable allosteric activity across the tested molecules. Notably, the N-terminal stearic modification of the binding motif YPWFYL exhibited a negative allosteric effect of −21.27% at a logEC50 (M) value of −6.52, showcasing its ability to modulate MOR signaling effectively. The table below provides additional data on the binding motifs (Candidates 1 and 2) and the resulting peptidomimetics (Candidate 3), offering a detailed comparison of their activity. The percentage values are normalized such that 0% corresponds to the activity of the reference agonist at EC20, while 100% represents the maximum activity of the reference agonist.

These experimental results confirm the capacity of the peptidosteroids to function as allosteric modulators of MOR, providing a strong foundation for their potential therapeutic application. Their ability to influence receptor activity selectively, combined with their tailored structural modifications, highlights the innovation and promise of this class of bioactive molecules.

| | | Allosteric activity | |
|---|---|---|---|
| Compound ID | logEC50(M) | level (%) | agonist present |
| sequence-1 | −5.34 | 23.64 | DAMGO |
| (Candidate 2) | | | |
| sequence-4 | −5.87 | 24.64 | DAMGO |
| (Candidate 1) | | | |
| sequence-1 | −6.00 | 21.67 | morphine |
| (Candidate 1) | | | |
| sequence-4 with | −6.52 | −21.27 | morphine |
| stearic acid N- | | | |
| Terminal | | | |
| modification | | | |
| (Candidate 3) | | | |

FIG. 1 illustrates a key and unexpected feature of the aspects of the disclosed embodiments by presenting the dose-activity relationship for one of the interaction motifs. The data reveals an EC50 value within the sub-5 range of logEC50 (M), signifying the required potency levels even at low concentrations. This high level of efficiency is attributed to the unique intramembrane activity of the peptidosteroid, a defining characteristic of its design. By confining the molecule to a limited spatial region of the lipid bilayer, the intramembrane localization increases its effective concentration near the mu-opioid receptor (MOR), enabling more efficient receptor engagement and signaling modulation.

In contrast to traditional ligands that diffuse freely through the aqueous environment, the intramembrane positioning of the peptidosteroid ensures that it remains in close proximity to its target receptor, amplifying its interaction potential. This spatial confinement significantly enhances their effective concentration near the target receptor, facilitating more efficient interactions with the mu-opioid receptor's allosteric sites. This intramembrane localization not only increases binding efficiency but also stabilizes receptor conformations that promote therapeutic signaling. The observed EC50 values reflect the improved efficacy of these molecules in modulating MOR activity at low concentrations, highlighting their potential for achieving analgesic effects with minimal doses. This efficiency further reduces the risk of systemic side effects, offering a distinct advantage over conventional opioid therapies. By harnessing the intramembrane activity of the peptidosteroids, the aspects of the disclosed embodiments set a new standard in the design of receptor-targeted therapeutics, demonstrating both superior efficacy and improved safety.

This unexpected property underscores the advanced engineering of the peptidosteroid and highlights its potential as a highly effective therapeutic agent. The combination of high potency, targeted activity, and selective receptor engagement not only validates the design principles underlying the aspects of the disclosed embodiments but also provides a foundation for improved safety profiles and reduced dosing requirements. By minimizing systemic exposure and associated side effects, the peptidosteroid represents a significant advancement in receptor-targeted drug development, particularly in the context of opioid-based therapies.

The water solubility of the peptidosteroids was experimentally verified with a standard protocol for Qualitative Solubility Test (at 0.1M PBS (pH 7.4±0.1), with DMSO presolubilisation). The water solubility of the invented molecules is an unexpected and significant element of the aspects of the disclosed embodiments, given the inherently hydrophobic nature of their modified residues. The ability to balance hydrophobicity with aqueous solubility is rare among membrane-interacting molecules and is a direct result of the deliberate structural design of the peptidosteroids. This property has significant implications for their pharmaceutical development, as it enables the formulation of these molecules in aqueous-based delivery systems, such as injectable solutions, without the need for excessive solubilizing agents.

This property is attributed to their unique conformations in aqueous solutions, which were thoroughly investigated using computational methods. To construct the starting states for molecular dynamics simulations and assess their structural properties, three-dimensional modeling of each peptidosteroid was performed in a water environment. Predicting the conformations of these molecules posed a challenge due to their specific amino acid compositions and motifs, which often lead to low structural organization and high conformational flexibility.

By employing tools such as Molecular Dynamics performed with dedicated software, and proprietary structure-prediction tools, we were able to predict probable solution-phase conformations and evaluate them using integrated scoring functions. The computational analysis revealed that these peptidosteroids adopt unique folded structures in aqueous environments, where hydrophobic residues are shielded by flexible hydrophilic segments. This folding behavior is believed to play a critical role in maintaining their solubility, allowing the peptidosteroids to remain stable and functionally active in physiological systems. The ability of these molecules to balance hydrophobicity with solubility through such distinct conformations represents a key and unexpected advancement in their design.

The peptidosteroids exhibit several critical advantages over traditional opioid therapies. By targeting allosteric sites and stabilizing receptor conformations associated with analgesia, these molecules mitigate the risks of addiction and other adverse effects. The incorporation of covalent modifications enhances their stability and bioavailability, allowing for lower dosing and extended dosing intervals. Furthermore, their design ensures a high degree of receptor selectivity, reducing the likelihood of off-target interactions and associated side effects.

Indeed, the allosteric modulators maintain the natural spatiotemporal signalling rhythms of orthosteric ligands when administered (5,6). Additionally, cooperation between binding allosteric and orthosteric ligands may lead to so called biased signalling, which promotes certain biological responses that may be desirable in therapeutic usage of the molecule (5,19). Allosteric drugs are also expected to present better safety profile in overdose situations due to weak cooperativity of target leading to “effect ceiling” (5,7,8).

The hypothesized mechanism of action involves a multi-step process. Upon administration, the peptidosteroids cross the water-bilayer interface, bind to the plasma membrane and undergo conformational changes that expose their binding motif. This motif interacts with the designated allosteric site on MOR, stabilizing the receptor in an extended conformation for analgesic signaling. Computational analyses suggest that this process involves intermediate states, which are critical for effective receptor engagement. These insights provide a framework for understanding the unique properties of peptidosteroids and guide the development of experimental validation protocols.

For example, sequences such as {ORN (3BETA-HYDROXY-5-CHOLENOICACID)} IVYAYL demonstrate optimized hydrophobicity and structural compatibility with MOR's allosteric binding pocket. These sequences were validated through computational docking and molecular dynamics simulations, confirming their ability to interact selectively with the receptor.

The structural features of the peptides were investigated using a variety of molecular modelling methods. We intended to investigate the possible conformations of the designed peptides both in aqueous solution and when bound to the target receptor. To generate high quality models and trajectories of possible peptide conformations we employed both knowledge-based approaches to folding and docking, as well as coarse-grained molecular dynamics. The analysis of results leads to conclusion that the peptides are expected to preferably adopt a folded conformation in aqueous solution and extended conformation when inserted into the bilayer or while interacting with the allosteric site of MOR.

The described peptidosteroids offer a novel approach to pain management by combining the benefits of allosteric modulation with the precision of peptide-based drug design. These molecules address the limitations of current opioid therapies, providing a safer and more effective alternative for chronic pain treatment. By reducing the risks associated with addiction, tolerance, and respiratory suppression, the aspects of the disclosed embodiments has the potential to significantly improve the quality of life for patients while addressing critical public health challenges associated with opioid misuse.

These peptidosteroids represent an improvement over existing therapies in their ability to safely modulate the mu-opioid receptor. By functioning as a highly specialized type of transmembrane allosteric modulator, they offer a safer alternative to traditional opioid therapies. Their design focuses on reducing the side effects commonly associated with opioid use, such as addiction and tolerance, while maintaining analgesic efficacy. This makes them particularly suitable for long-term management of chronic pain.

The safety profile of these peptidosteroids is a critical aspect of their design. Through selective interaction with the MOR and their allosteric modulation capabilities, they are expected to produce fewer side effects compared to conventional opioids.

The safety profile of the peptidosteroids was evaluated through a series of exploratory toxicity tests conducted on cell cultures, followed by subsequent in vivo assessments in mice. Initial in vitro studies utilized mammalian cell lines to investigate cytotoxic effects at various concentrations of the peptidosteroids. These tests demonstrated negligible impact on cell viability, even at concentrations above the predicted therapeutic range, suggesting a favorable safety margin. Building on these results, the molecules were further tested in vivo using standard toxicity evaluation protocols in murine models. The mice exhibited no adverse effects, with no evidence behavioral abnormalities during the observation period. These findings align closely with the predictions from our in silico modeling, which suggested that the peptidosteroids would exhibit minimal off-target interactions or destabilizing effects on cellular membranes.

This validation is particularly significant given the inherent risks associated with membrane-binding and interacting molecules, which often display potential for cytotoxicity due to their ability to disrupt lipid bilayer integrity or perturb critical membrane-associated processes. The non-toxic nature of the peptidosteroids underscores the precision of their design, ensuring selective interaction with the mu-opioid receptor without adversely affecting other cellular components. This represents a critical advancement, as it not only supports the viability of these molecules as therapeutic agents but also highlights their unique capability to achieve membrane-specific targeting while maintaining an exceptional safety profile. Such properties position the peptidosteroids as a promising class of compounds for clinical development, addressing key challenges in receptor-targeted drug design.

The peptidosteroids described herein offer a novel approach to chronic pain management. By combining advanced computational design techniques with innovative synthesis processes, the aspects of the disclosed embodiments provide a safer, more effective alternative to existing opioid-based therapies. These peptidosteroids hold the potential to transform the treatment landscape for chronic pain, offering hope for patients seeking relief with minimal side effects.

What is claimed is:

1. A short peptide sequence that allosterically modulates the activity of the human mu-opioid receptor (MOR) via binding to the transmembrane region, wherein said peptide sequence is designed for eliciting analgesic effects.

2. The short peptide sequence of claim 1, wherein the peptide comprises natural amino acids, D-amino acids, retro-inverted sequences, or a combination thereof.

3. The short peptide sequence of claim 1, wherein the peptide is designed for entropy-driven binding to the transmembrane region of the MOR.

4. The short peptide sequence of claim 1, wherein the peptide is designed for preferential activation of a desired intracellular signaling pathway while limiting adverse effects due to other pathway activation.

5. The short peptide sequence of claim 1, incorporating N-terminal modifications including acylation, lipid derivatives of varying polycarbon chain lengths up to palmitoyl modification, or C-terminal modifications including amidation.

6. A peptidosteroid compound comprising a specific amino acid sequence modified for selective interaction with the MOR, wherein the compound includes covalent modification with 3beta-hydroxy-5-cholenoic acid, decanoic acid, lauric acid, myristoyl, palmitoyl, or stearic acid to enhance its stability, membrane affinity, intramembrane localization, and receptor selectivity.

7. The short peptide sequence of claim 1 or the peptidosteroid compound, for use in inducing analgesic effects with reduced side effects, suitable for the treatment of chronic pain conditions selected from the group consisting of neuropathic pain, cancer-related pain, and postoperative pain.

8. A pharmaceutical composition comprising one or more peptides or peptidosteroids according to claim 1 and a pharmaceutically acceptable carrier, wherein the composition is optimized for oral, injectable, or transdermal delivery.

9. The pharmaceutical composition of claim 8, further comprising a reference agonist of the MOR or additional therapeutic agents, including orthosteric MOR ligands, for synergistic treatment of chronic pain or related disorders.

10. A method of treating chronic pain in a subject, comprising administering an effective amount of the pharmaceutical composition according to claim 8.

11. The method of claim 10, wherein the chronic pain condition is selected from the group consisting of neuropathic pain, cancer-related pain, and postoperative pain.

12. The peptidosteroid compound of claim 6, wherein the interaction with the MOR is mediated through intramembrane binding sites, facilitated by the compound's amphiphilic properties and affinity for the plasma membrane.

13. The peptidosteroid compound of claim 6, wherein the compound adopts a conformation that transitions between folded and extended states to optimize solubility in aqueous environments and receptor binding in lipid bilayers.

14. A method for enhancing MOR modulation, comprising the co-administration of the pharmaceutical composition of claim 10 with an orthosteric MOR ligand, wherein the peptide or peptidosteroid functions as an allosteric modulator to bias signaling pathways toward therapeutic outcomes.

15. A method for identifying candidate peptides or peptidosteroids for therapeutic applications, comprising computational modeling to predict binding affinities, receptor interactions, and pharmacokinetic properties, followed by experimental validation.

16. Use of the peptides or peptidosteroids of claim 1 in combination with orthosteric opioid agonists, wherein the peptides or peptidosteroids modulate receptor activity to reduce the required dose of the orthosteric agonist, thereby minimizing associated side effects.
